(19) United States
(12) Patent Application Publication
(10) Pub. No.: US 2003/0216479 A1
(43) Pub. Date: Nov. 20, 2003

(54) Novel compositions comprising 2,2-Bis (4-hydroxy-3-methylphenyl) heptane and uses thereof

(76) Inventors: Liren Huang, Rockville, MD (US); Alenka Tomazic, Ljubljana (SI); Joanna Clancy, Rockville, MD (US); Vito M. Esposito, Potomac, MD (US)

Correspondence Address: MANELLI DENISON & SELTER, 2000 M STREET NW SUITE 700, WASHINGTON, DC, 20036-3307, US

(21) Appl. No.: 10/289,297

(22) Filed: Nov. 7, 2002

Related U.S. Application Data
(60) Provisional application No. 60/331,096, filed on Nov. 8, 2001.
(60) Provisional application No. 60/406,322, filed on Aug. 28, 2002.

(51) Int. Cl. A61K 31/075; A61K 31/375

(52) U.S. Cl. 514/721; 514/474

(57) ABSTRACT

The present invention is drawn to anti-microbial compositions comprising 2,2-Bis(4-hydroxy-3-methylphenyl)heptane and use of the compositions.

The present application claims priority on U.S. Provisional Application No. 60/331,096, filed Nov. 8, 2002 and U.S. Provisional Application No. 60/406,322, filed, Aug. 28, 2002, both of which are hereby incorporated by reference.

### FIELD OF INVENTION

The present invention is directed to antimicrobial compositions comprising 2,2-Bis(4-hydroxy-3-methylphenyl)heptane and use of the compositions. The antimicrobial compositions of the invention are effective in killing or inhibiting a wide variety of harmful, destructive, pathogenic or offensive microorganisms, including viruses, bacteria, protozoa, algae and fungi, including dormant and/or resistant forms such as spores and cysts. One aspect of the invention is a composition for the treating of surfaces to remove, reduce or retard the growth or activity of microorganisms. Another aspect of the invention is a composition that is effective in treating or preventing an infection in an animal or plant. The present invention is also directed to devices that are coated with compositions comprising 2,2-Bis(4-hydroxy-3-methylphenyl)heptane and methods of coating the devices.

### BACKGROUND OF THE INVENTION

There are many known uses for antimicrobial agents as antiseptics and for cleaning, disinfecting and sterilizing, and for preventing and treating many diseases. Because microorganisms are often able to develop resistant strains to antimicrobial agents, there is a continual need for the development of new antimicrobial compositions employing such compounds. Such compositions are used, for example, in the health care industry, food service industry, meat processing industry, in the workplace and in the home.

Bacteria, fungi, protozoa, viruses, algae and other microorganisms are always present in the environment and may be undesirable because they cause illness or death of humans and animals, create odors and damage or destroy a wide variety of materials. The species and numbers of microorganisms present vary depending on the characteristics of the particular environment, i.e., the nutrients, moisture and temperature. For example, surfaces including walls, floors, food preparation surfaces, instruments and devices may become contaminated by fungal, protozoan, algal, bacterial or viral microorganisms. Furthermore, many organic, synthetic and natural materials such as plastic coatings and objects, wood, paper and natural fibers serve as nutrients for microorganisms and may be degraded by the microorganisms. Additionally certain microorganisms are capable of remaining viable in a dormant state on floors or on objects for long periods of time until they are deposited in a proper environment for growth. As a result, microorganisms can be spread by walking on floor, brushing against walls or furniture or by handling objects (e.g., towels, instruments).

Under the appropriate conditions, microbes have a tendency to adhere to surfaces and initiate the formation of a biofilm. A biofilm is a community of microbes adhering to an inert or living surface. Microbes growing in biofilms are more resistant to antibiotics and disinfectants than planktonic cells and the resistance increases with the age of the biofilm. Biofilms can form on virtually any surface including contact lenses, ship hulls, pipelines, rocks, biomedical devices and implants, and mucous surfaces (Dunne, Clinical Microbiological Reviews, 15, 155, 2002). Biofilms cause a variety of problems, including, but not limited to, increased frictional resistance to fluids in water conduits, decreased heat transfer from heat exchanges, pneumoniae, otitis media, dental plaque, and contamination of medical devices such as endoscopes, catheters, prosthetic devices and medical implants.

Several different classes of agents have been used in antimicrobial compositions, including, bisguanidine, di-phenyl compounds, benzyl alcohols, trihalocarbanilides, quaternary ammonium compounds, exthoxlated phenols and phenolic compounds such as halo-substituted phenolic compounds like p-chloro-m-xylenol and triclosan (U.S. Pat. No. 6,136,771). Phenolics such as triclosan are a class of antimicrobial agents which have been demonstrated to have broad spectrum activity against a variety of microorganisms (WO 99/51094 and Domagala et al. in Resolving the Antibiotic Paradox, ed by Rosen and Mabashery, page 269, 1998). While Domagala et al. teach some diphenolic methane derivatives as having in vitro anti-bacterial activity, Domagala et al. teaches that these compounds do not have in vivo anti-bacterial activity when administered orally or subcutaneously. This is in sharp contrast to compositions of the present invention which surprisingly, do have in vivo anti-bacterial activity and other antimicrobial activity when administered topically or subcutaneously.

The increasing prevalence of bacteria and other microbes that are resistance to existing antimicrobial agents necessitates the identification of new classes of antimicrobials. Accordingly there is a need both in industry and in the home for safe and effective antimicrobial compositions which can be used as antimicrobials in or on a wide variety of substances and surfaces to reduce or eliminate microorganisms and which can be used to therapeutically or prophylactically treat animals, plants or inanimate objects.

None of the prior teachings, described above or elsewhere, disclose compositions comprising 2,2-Bis(4-hydroxy-3-methylphenyl)heptane as an effective antimicrobial agent. It is therefore an object of this invention to provide compositions comprising 2,2-Bis(4-hydroxy-3-methylphenyl)heptane that are useful as agents for the treatment of bacterial, viral, protozoan, or fungal infections both in vivo (including but not limited to parenterally and topically) and for inhibiting bacterial, viral, protozoan, algal or fungal growth, for example on surfaces, in biofilms, or in liquids.

### SUMMARY OF THE INVENTION

The present invention includes antimicrobial compositions comprising 2,2-Bis(4-hydroxy-3-methylphenyl)heptane and use of the compositions. The invention encompasses prophylactic and/or therapeutic compositions comprising 2,2-Bis(4-hydroxy-3-methylphenyl)heptane and methods of treating or preventing disease including but not limited to acne, skin infections, cutaneous anthrax, pneumonia, diabetic ulcers, pressure ulcers, soft tissue infections, burns, paronychia, mucosal infection, ophthalmic infections and more particularly diseases associated with infection by Staphylococcal spp., Streptococcus spp, Haemophilus spp., Moraxella, Candida albicans, Helicobacter pylori, Bacillus anthracis, Chlamydia pneumoniae, Chlamydia trachomatis, Neisseria gonorrhea, Neisseria meningitidis, HIV, Trichomonas vaginitis, or Giardia, or Trichophyton spp.

In a preferred embodiment the composition further comprises one or more agents selected from the group consisting of ascorbic acid, ascorbate, zinc chloride, chelating agents, nonionic and anionic surfactants, polyols, gelling agents, hydrotopes, pH adjusters, skin conditioners, buffers, polyethylene glycols, dyes, aqueous carriers, chlorhexidine, and alcohols.

In another preferred embodiment, the composition comprises triclosan or a derivative of triclosan and additionally 2,2-Bis(4-hydroxy-3-methylphenyl)heptane. In another preferred embodiment, the composition comprises 2,2-Bis(4-hydroxy-3-methylphenyl)heptane and a lantibiotic. In another preferred embodiment, the composition comprises 2,2-Bis(4-hydroxy-3-methylphenyl)heptane and mupirocin or a pharmaceutically acceptable ester salt. In another preferred embodiment, the composition comprises 2,2-Bis(4-hydroxy-3-methylphenyl)heptane and EDTA (ethylenediaminetetra acetic acid).

The invention also encompasses compositions for treating a surface to remove, reduce or inhibit the growth or activity of microorganisms on the surface. Specific uses for the compositions include cleaners for hospitals, nursing homes, daycare centers, dentists' office, and doctors' offices, coatings for gloves and clothing, sprays to kill microorganisms (e.g., bacteria, fungi, protozoa, algae, or viruses), deodorants, foot powders, insoles for shoes, paper tissues, wet wipes, sponges, hand sanitizers, detergents, topical lotions, cosmetics, air duct filters, bandages, containers which carry liquid or bulk food including trucks, cleaners for showers and toilets, humidifiers, teat dips for dairy cows, salves for sores, carpet shampoos, cleaners for medical, dental or laboratory instruments, preservatives for food, chewing gums, lozenges, toothpaste additives and mouthwashes. The invention also provides methods for cleaning and disinfecting a surface at least partly covered by a biofilm comprising contacting the biofilm with a composition comprising 2,2-Bis(4-hydroxy-3-methylphenyl)heptane. The present invention also includes a method of cleaning and disinfecting an inert or living surface at least partly covered by a biofilm layer by contacting the biofilm with a composition comprising 2,2-Bis(4-hydroxy-3-methylphenyl)heptane in an amount effective for either fully or partly removing or releasing the biofilm layer. The present method also includes a method of inhibiting the formation of a biofilm on a surface comprising contacting a surface with a composition comprising 2,2-Bis(4-hydroxy-3-methylphenyl)heptane.

An object of the invention is to provide antimicrobial agents and compositions that are applied to plants by any of a variety of art-recognized means. For example, the compositions can be applied to the plants' surface by spraying. Alternatively, the solution can be introduced injectably into a plant, for example, with a syringe, applied as a solid fertilizer-like preparation for absorption by the roots at the base of a plant or a solution can be distributed at the base of a plant for root absorption.

It is also an object of the invention to provide compositions for and a method for removing or killing microorganism from animal carcasses and fresh meat products using solutions comprising 2,2-Bis(4-hydroxy-3-methylphenyl)heptane.

It is also an object of the invention to provide a method of coating surfaces such as those of a device, including, medical and laboratory devices, comprising the steps of applying to at least a portion of the surface of the device an antimicrobial coating layer comprising 2,2-Bis(4-hydroxy-3-methylphenyl)heptane in a effective concentration to inhibit the growth or activity of microorganisms (e.g., bacterial, protozoan, fungi, algae, or viruses) and optionally applying one or more resilient protective coatings over the antimicrobial coating layer. Medical devices amenable to treatment by the present invention, include but are not limited to, urinary catheters, vascular catheters, wound drainage tubes, shunts, cannulas, arterial grafts, soft tissue patches, gloves, stents, tracheal catheters, respirators, wound dressings, sutures, guide wires and prosthetic devices.

### BRIEF DESCRIPTION OF THE FIGURES

FIG. 1. Efficacy of a composition comprising 2,2-Bis(4-hydroxy-3-methylphenyl)heptane in the wound healing model. A defined inoculum of approximately 10⁵ CFU of methicillin-sensitive Staphylococcus aureus in 20 μl culture medium was pipetted into a wound approximately 1 cm² created by removal of the epidermis from the shaved backs of mice. After 24 hours, the composition comprising 2,2-Bis(4-hydroxy-3-methylphenyl)heptane, placebo control or positive control ointment was applied twice daily. Approximately, seventy two hours post-infection, the wound was sampled and appropriate dilutions plated on nutritive agars for determination of microbial burden. The number of colony forming units (CFU) of methicillin-sensitive Staphylococcus aureus per wound is shown.

FIG. 2. Efficacy of a composition comprising 2,2-Bis(4-hydroxy-3-methylphenyl)heptane in the wound healing model. A defined inoculum of approximately 10⁵ CFU of methicillin-resistant Staphylococcus aureus in 20 μl culture medium was pipetted into a wound approximately 1 cm² created by removal of the epidermis from the shaved backs of mice. After 24 hours, the composition comprising 2,2-Bis(4-hydroxy-3-methylphenyl)heptane, placebo control or positive control ointment was applied twice daily. Approximately, seventy two hours post-infection, the wound was sampled and appropriate dilutions plated on nutritive agars for determination of microbial burden. The number of colony forming units (CFU) of methicillin-resistant Staphylococcus aureus per wound is shown.

FIG. 3. Efficacy of a composition comprising 2,2-Bis(4-hydroxy-3-methylphenyl)heptane in the wound healing model. A defined inoculum of approximately 10⁵ CFU of methicillin-sensitive Staphylococcus epidermis in 20 μl culture medium was pipetted into a wound approximately 1 cm² created by removal of the epidermis from the shaved backs of mice. After 24 hours, the composition comprising 2,2-Bis(4-hydroxy-3-methylphenyl)heptane, placebo control or positive control ointment was applied twice daily. Approximately, seventy two hours post-infection, the wound was sampled and appropriate dilutions plated on nutritive agars for determination of microbial burden. The number of colony forming units (CFU) of methicillin-sensitive Staphylococcus epidermis per wound is shown.

FIG. 4. Effect of 2,2-Bis(4-hydroxy-3 methylphenyl)heptane on biofilm formation by Staphylococcus aureus 43300. A biofilm-enriched variant of S. aureus 43300 (methicillin-resistant) forms stable biofilms on the surface of fibronectin-treated coverslips after 8 hours of incubation with liquid bacterial cultures. Biofilms on coverslips are rinsed and incubated in TSB +1% glucose with antibiotics at 64 mg/L for an additional 18 hours. The viable cells on the biofilm are harvested and enumerated by dilution and plating to determine colony forming units.

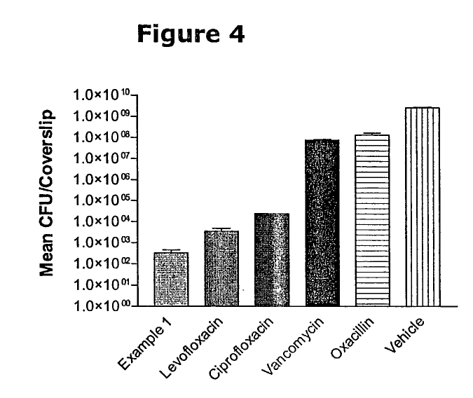

FIG. 5. Efficacy of a composition comprising 2,2-Bis(4-hydroxy-3-methylphenyl)heptane in the wound healing model. A defined inoculum of approximately 105 CFU Streptococcus pyrogenes in 20 μl culture medium was pipetted into a wound approximately 1 cm² created by removal of the epidermis from the shaved backs of mice. After 24 hours, the composition comprising 2,2-Bis(4-hydroxy-3-methylphenyl)heptane, placebo control or positive control ointment was applied twice daily. Approximately, seventy two hours post-infection, the wound was sampled and appropriate dilutions plated on nutritive agars for determination of microbial burden. The number of colony forming units (CFU) of Streptococcus pyrogenes per wound is shown.

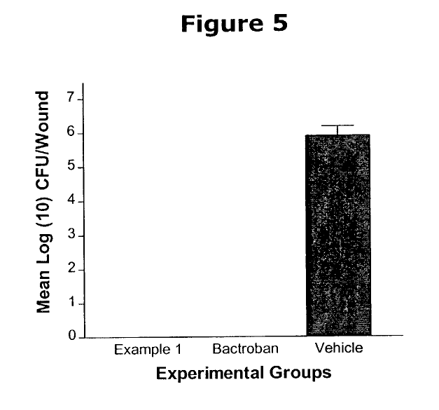

### DETAILED DESCRIPTION OF THE INVENTION

The present invention is directed to antiseptic or disinfectant compositions of the invention comprising 2,2-Bis(4-hydroxy-3-methylphenyl)heptane.

The term disinfectant is used herein to mean a composition applied to inanimate objects used to destroy, reduce or retard the growth, activity or infectivity of microorganisms, including but not limited to bacteria, fungi, protozoa, algae, and viruses on the inanimate objects. The term antiseptic is used herein to mean a composition applied to animals or plants that destroys inhibits or stops the growth, activity or infectivity of microorganisms including but not limited to bacteria, fungi, protozoa, algae, and viruses, and in one embodiment is used to treat or prevent a disease caused by or associated with an infectious microorganism. The term antimicrobial is used herein to mean a composition used as an antiseptic and/or a disinfectant.

In a preferred embodiment, the antiseptic composition comprises about 0.05% to about 25%, preferably about 0.5% to 25% and most preferably about 0.5% to 20% of 2,2-Bis(4-hydroxy-3-methylphenyl)heptane. The compositions also can be formulated as concentrates that are diluted before use.

In a preferred embodiment, the disinfectant composition comprises about 5% to about 50%, preferably about 15% to 50% and most preferably about 15% to 40% of 2,2-Bis(4-hydroxy-3-methylphenyl)heptane. The compositions also can be formulated as concentrates that are diluted before use.

In a preferred embodiment both the antiseptic and disinfectant compositions further comprise one or more agents selected from the group consisting of zinc chloride, ascorbic acid, ascorbate, nonionic and anionic surfactants, alcohols, chelators, polyols, gelling agents, hydrotopes, pH adjusters, skin conditioners, chlorhexidine, dyes and fragrances. The agents are present in a sufficient amount to perform their intended function and do not adversely affect the antimicrobial efficacy of the composition.

Suitable surfactants are those which are reasonably stable and preferably form suds through the pH range of the compositions. Surfactants useful as sudsing agents may be soaps, and anionic, nonionic, cationic, zwitterionic and amphoteric organic synthetic detergents, and compatible mixtures thereof. Surfactants of these types are described more fully in U.S. Pat. No. 3,959,458 issued to Agricola, Briner, Granger & Widder on May 25, 1976 and U.S. Pat. No. 3,937,807 issued to Haefele on Feb. 10, 1976, both of which are incorporated herein by reference in their entirety. Such surfactants are generally present in the compositions of the subject invention at a level of from about 0% to about 10%. Surfactants may also be used as solubilizing agents to help retain sparingly soluble components, e.g., some flavoring agents, in solutions. Surfactants suitable for this purpose include polysorbates and poloxamers.

Suitable chelators include, but are not limited to EDTA, CaEDTA, CaNa₂EDTA, EGTA (ethylene glycol bis(β-aminoethylether)N,N,N′,N′-tetra acetic acid) and citrate.

Suitable alcohols include, but are not limited to, propylene glycol, 1,3-propanediol, 1,2-butanediol, PEG (polyethylene glycol) 200, PEG 400, PEG 600, PEG 900, PEG 3350, PEG 1450, PEG 6000, glycerol, 1,4 butanediol, C₁₋₆ alcohols e.g., ethanol and isopropanol, methanol, n-butyl alcohol, tert-butyl alcohol, or mixtures thereof.

The following are non-limiting examples of gelling agents that can be used in the present invention. In particular, the following compounds both organic and inorganic, act primarily by thickening or gelling the aqueous portion of the composition: acacia, acrylates/steareth-20 methacrylate copolymer, agar, algin, alginic acid, ammonium chloride, ammonium sulfate, amylopectin, bentonite, C₉₋₁₅ alcohols, calcium acetate, calcium alginate, calcium carrageenan, calcium chloride, caprylic alcohol, carbomer 910, carbomer 934, carbomer 934P, carbomer 940 carbomer 941, carboxymethy hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylenedioleamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA (monoisopropanolamine), hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystallinc cellulose, montmorillonite, myristyl alcohol, oat flour, oleyl alcohol, palm kernel alcohol, pectin, PEG-200, PEG-600, polyacrylic acid, polyvinyl alcohol, potassium alginate, potassium aluminum polyacrylate, potassium carrageenan, potassium chloride, potassium sulfate, potato starch, propylene glycol alginate, sodium acrylate/vinyl alcohol copolymer, sodium carboxymethyl dextran sodium carrageenan, sodium cellulose sulfate, sodium chloride, sodium polymethacrylate, sodium silicoaluminate, sodium sulfate, stearalkonium bentonite, stearalkonium hectorite, stearyl alcohol, tallow alcohol, TEA (triethanolamine)-hydrochloride, tragacanth gum, tridecyl alcohol, trimethamine magnesium aluminum silicate, wheat flour, wheat starch, xanthan gum, and mixtures thereof.

The following additional non-limiting examples of gelling agents act primarily by thickening the nonaqueous portion of the composition: abietyl alcohol, acrylinoleic acid, aluminum behenate, aluminum caprylate, aluminum dilinoleate, aluminum distearate, aluminum isostearates/laurates/palmitates or stearates, aluminum isostearates/myristates, aluminum isostearates/palmitates, aluminum isostearates/stearates, aluminum lanolate, aluminum myristates/palmitates, aluminum stearate, aluminum stearates, aluminum tristearate, beeswax, behenamide, behenyl alcohol, butadiene/acrylonitrile copolymer, calcium behenate, calcium stearate, candelilla wax, carnauba, ceresin, cholesterol, cholesteryl hydroxystearate, coconut alcohol, copal, diglyceryl stearate malate, dihydroabietyl alcohol, dimethyl lauramine oleate, dodecanedioic acid/cetearyl alcohol/glycol cqpolymer, erucamide, ethylcellulose, glyceryl triacetyl hydroxystearate, glyceryl tri-acetyl ricinoleate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable glycerides, hydrogenated vegetable oil, hydroxypropylcellulose, isobutylene/isoprene copolymer, isocetyl stearoyl stearate, Japan wax, jojoba wax, lanolin alcohol, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydride copolymer, octyldodecyl stearoyl stearate, oleamide, oleostearine, ouricury wax, oxidized polyethylene, ozokerite, palm kernel alcohol, paraffin, pentaerythrityl hydrogenated rosinate, pentaerythrityl rosinate, pentaerythrityl tetraabietate, pentaerythrityl tetrabehenate, pentaerythrityl tetraoctanoate, pentaerythrityl tetraoleate, pentaerythrityl tetrastearate, phthalic anhydride/glycerin/glycidyl decanoate copolymer, phthalic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP (polyvinylpyrrolidone)/eicosene copolymer, PVP/hexadecene copolymer, rice bran wax, stearalkonium bentonite, stearalkonium hectorite, stearamide, stearamide DEA (diethonolamine)-distearate, stearamide DIBA-stearate, stearamide MEA (monothanolamine)-stearate, stearone, stearyl alcohol, stearyl erucamide, stearyl stearate, stearyl stearoyl stearate, synthetic beeswax, synthetic wax, trihydroxystearin, triisononanoin, triisostearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, zinc stearate, and mixtures thereof.

The hydrotrope, if present at all, is present in an amount of about 0.1% to about 30%, and preferably about 0.5% to about 25%, by weight of the composition. A hydrotrope is a compound that has the ability to enhance the water solubility of other compounds. A hydrotrope lacks surfactant properties, and typically is a short-chain alkyl aryl sulfonate. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, and disodium succinate.

Basic pH adjusters include, but are not limited to, ammonia; mono-, di-, and tri-alkyl amines; mono-, di, and tri-alkanolamines; alkali metal and alkaline earth metal hydroxides; and mixtures thereof. However, the identity of the basic pH adjuster is not limited, and any basic pH adjuster known in the art can be used. Specific, nonlimiting examples of basic pH adjusters are ammonia; sodium, potassium, and lithium hydroxide; monoethanolamine; triethylamine; isopropanolamine; diethanolamine; and triethanolamine. acidic pH adjusters include but are not limited to the mineral acids and polycarboxylic acids. Nonlimiting examples of mineral acids are hydrochloric acid, nitric acid, phosphoric acid, and sulfuric acid. Nonlimiting examples of polycarboxylic acids are citric acid, glycolic acid, and lactic acid. The identity of the acidic pH adjuster is not limited and any acidic pH adjuster known in the art, alone or in combination, can be used.

Skin conditioners include but are not limited to, emollients, such as, cetyl myristate, glyceryl dioleate, isopropyl myristate, lanolin, methyl laurate, PPG (polypropylene glycol)-9 laurate, soy stearyl, octyl palmitate, and PPG-5 laurate, for example. The skin conditioner also can be a humectant, for example, glucamine and pyridoxine glycol, for example. Occlusive skin conditioners, for example, aluminum lanolate, corn oil, methicone, coconut oil, stearyl stearate, phenyl trimethicone, trimyristin, olive oil, and synthetic wax, also can be used. Combinations of the classes of skin conditioners, in addition to miscellaneous skin conditioners known to persons skilled in the art, alone or in combination can be used. Nonlimiting examples of miscellaneous skin conditioners include aloe, cholesterol, cystine, keratin, lecithin, egg yolk, glycine, PPG-12, retinol, salicylic acid, orotic acid, vegetable oil, and soluble animal collagen. The skin conditioners can be used alone, or in combination with a skin protectant, like petroleum, cocoa butter, calamine, and kaolin, for example. A skin protectant also can be used alone. Additional examples of skin conditioners and protectants can be found in “CTFA Cosmetic Ingredient Handbook,” J. M. Nikitakis, ed., The Cosmetic, Toiletry and Fragrance Association, Inc., Washington, D.C. (1988) (hereafter CTFA Handbook), pages 79-85, incorporated herein by reference.

Disinfecting a surface can be accomplished by any conventional method used to clean surfaces such as by means of a brush, cloth or sponge wetted with the treating solution or by immersion of the surface in the treating solution or contacting the surface with an automatic washer for a period of time sufficient to remove, retard or reduce contamination. Any surface may be treated, including but not limited to steel, wood, or plastic. The treating solution may be sprayed on as a wet or dry formulation. In some instances time release formulations may find use, particularly for applications to surface subject to recontamination.

It is also an object of the invention to provide a composition for and a method for removing or killing microorganisms on animal carcasses and fresh meat products using solutions comprising 2,2-Bis(4-hydroxy-3-methylphenyl)heptane. The solution (or other liquid carrier such as emulsion or suspension) of the invention is used as a dip or as a spray or as a combination of these applications. In a typical method for disinfecting poultry carcasses or poultry carcass pieces, for example, the carcasses or pieces are washed after evisceration, immersed in a chilling tank and then removed from the tank. An optional wash of the carcasses or pieces may follow these processing steps. In the practice of the invention, the carcasses or carcass pieces are contacted with the disinfectant solution during the initial washing after evisceration, during the chill tank immersion period or during a spray wash removal from the chill tank. The disinfectant solution of the invention may also be used in more than one of these processing steps. A food-grade wetting agent such as alkylphenoxypoly(ethylene oxide), a poly (ethylene oxide/propylene oxide)block copolymer, an alkylbenzene sulfonic acid, a dioctylsolfosuccinate and mixtures of these may be added to the solution to facilitate contact with the meat surfaces. The solution may also contain effective amounts of a food-grade thickener such as xanthum gum, or alginic acid preferably one sufficient to achieve a final solution (i.e., liquid) viscosity of from about 5 cps to about 50 cps at room temperature, for spray applications.

It is also an object of the invention to provide a method of coating a device comprising the steps of applying to at least a portion of the surface of the device an antimicrobial coating layer composition comprising 2,2-Bis(4-hydroxy-3-methylphenyl)heptane in a effective concentration to inhibit the growth of microorganisms (e.g. bacterial, fungi, protozoa, algae or virus) and optionally applying resilient protective coating over the antimicrobial coating layer.

When the device comprises plastic (or other resin), the antimicrobial composition comprises 2,2-Bis(4-hydroxy-3-methylphenyl)heptane may be incorporated into the plastic material in an effective concentration to inhibit the growth or activity of harmful microorganisms and to protect the plastic from degradation by harmful microorganisms. Devices composed of a variety of materials, e.g. metal, that are treated with the present compounds/compositions can be used to make a wide variety of products such as furniture, medical devices, laboratory equipment, tubing, food handling trays, shower curtains. The present compositions may be incorporated in the material from which the device is fabricated or may be used to treat the surface of the device. Medical devices encompassed by the present invention, include but are not limited to, urinary catheters, vascular catheters, drainage bags, colostomy pouches, ileostomy pouches, wound drainage tubes, arterial grafts, soft tissue patches, gloves, shunts, stents, tracheal catheters, wound dressings, sutures, guide wires microsphere, condoms, dental floss, microspheres, and prosthetic devices.

Disinfectant compositions comprising 2,2-Bis(4-hydroxy-3-methylphenyl)heptane can be applied topically to fabrics, to natural and synthetic fibers or can be incorporated directly onto synthetic fibers to impart antimicrobial activity to the fibers or fabrics made from the fibers. Fiber or fabric products include but are not limited to air filters, surgical gauze, padding on wound dressings, mattress covers, crib covers, sailboat sails, tents, draw sheets, and hospital clothing such as physician coats and nurse uniforms.

The compositions of this invention can be administered as treatment for bacterial, protozoan, viral, or fungal infections or colonization (including biofilms comprising one or more microbes) by any means that produces contact of the active agent with the bacteria, protozoa, virus or fungus in the living body of an animal or plant or on the surface of animal carcasses or nonliving objects. They can be administered by any conventional means available for use in conjunction with pharmaceuticals, either as individual therapeutic agents or in a combination of therapeutic agents.

The present compound can be administered alone, but is generally administered with a pharmaceutical carrier selected on the basis of the chosen route of administration and standard pharmaceutical practice. In a preferred embodiment the pharmaceutical carrier is slightly polar. In a more preferred embodiment, the formulation comprises 5-35% 2,2-Bis(4-hydroxy-3-methylphenyl)heptane in PEG 600, PEG 400, or PEG 200. In another preferred formulation, the formulation comprises 5-35% 2,2-Bis(4-hydroxy-3-methylphenyl)heptane in 10-40% PEG3350/60-90% PEG 400. In yet another preferred formulation, the formulation comprises 5-35% 2,2-Bis(4-hydroxy-3-methylphenyl)heptane in 5-35% PEG 8000/65-95% PEG200 In another preferred formulation, the formulation comprises 5-35% 2,2-Bis(4-hydroxy-3-methylphenyl)heptane in 10-45% propylene glycol/50-75% ethanol or isopropanol. In another preferred embodiment the formulation comprises 5-35% 2,2-Bis(4-hydroxy-3-methylphenyl)heptane in PEG 200/0.1-1% ascorbate. In another preferred embodiment, the composition comprises 2,2-Bis(4-hydroxy-3-methylphenyl)heptane and benzoyl peroxide. In another preferred embodiment the composition comprises 2,2-Bis(4-hydroxy-3-methylphenyl)heptane and boric acid. In another preferred embodiment the composition comprises 2,2-Bis(4-hydroxy-3-methylphenyl)heptane and polyvinyl pyrrolidone.

The compositions of the invention are of course given by forms suitable for each administration route. For example, they are administered in tablets or capsule form, by injection, inhalation, eye lotion, ointment, foams, suppository, etc., and administered by infusion, topically, vaginally or rectally. Oral or topical administration is preferred. The compositions of the invention are useful for the treatment of infections in hosts, especially mammals, including humans, in particular in humans and domesticated animals (including but not limited to equines, cattle, swine, sheep, poultry, feline, canine and pets in general) and plants. The compositions may be used, for example, for the treatment of infections of skin, mouth, eye, the respiratory tract, the urinary/reproductive tract, and soft tissues and blood, especially in humans.

2,2-Bis(4-hydroxy-3-methylphenyl)heptane may be used in combination with one or more therapeutic partners for the treatment of infections. The term “therapeutic partner” or “therapeutic agent” as used herein and in the claims includes but is not limited to antibiotic (for example, tobramycin, cephalosporin), steroids, vaccines, anti-oxidants, zinc chloride, ascorbic acid, ascorbate, dextran sulfate, non-steroidal anti-inflammatories, antacids, antibodies, chelators, interferons, protease, glucosidases, pectinase, amylase, lipase and/or cytokines.

Examples of therapeutic partners that may be administered with 2,2-Bis(4-hydroxy-3-methylphenyl)heptane include, but are not limited to nystatin, amphotericin B, griseofulvin, flucytosine, potassium iodide, polymixin B, imidazole (i.e. clotrimazole, miconazole, ketoconazole), tolnaftrate, cyclophosamide, ascorbic acid, ascorbate, meprazole, dextran sulfate, bismuth, histamine H2 receptor antagonist, chlorhexidine, mupirocin, serine protease, polyvinyl pyrrolidone, benzoyl peroxide, boric acid, glucosidase, lanthionine bacteriocins, clarithromycin, omeprazole, metronidazole, quinacrine hydrochloride, norfloxacin, ciprofloxacin, levofloxacin, enoxacin, triclosan, clarithromycin, tobramycin, cephalosporin, furazolidone, tiniadazole, tetracycline, Lansoprazole imipenem, meropenem, biapenem, aztreonam, latamoxef (MOXALACTAM™), and other known beta-lactam antibiotics, benzylpenicillin, phenoxymethylpenicillin, carbenicillin, azidocillin, propicillin, ampicillin, amoxycillin, epicillin, ticarcillin, cyclacillin, pirbenicillin, azlocillin, mezlocillin, sulbenicillin, piperacillin, and other known penicillins. The penicillins may be used in the form of pro-drugs thereof, for example as in vivo hydrolysable esters, for example the acetoxymethyl, pivaloyloxymethyl, alpha-ethoxycarbonyloxyethyl and phthalidyl esters of ampicillin, benzylpenicillin and amoxycillin; as aldehyde or ketone adducts of penicillins containing a 6-alpha-aminoacetamido side chain (for example hetacillin, metampicillin and analogous derivatives of amoxicillin); and as alpha-esters of carbenicillin and ticarcillin, for example the phenyl and indanyl alpha-esters. Cephalosporins that may be therapeutic partners with 2,2-Bis(4-hydroxy-3-methylphenyl)heptane, but are not limited to, cefatrizine, cephaloridine, cephalothin, cefazolin, cephalexin, cephacetrile, cephapirin, cephamandole nafate, cephradine, 4-hydroxycephalexin, cephaloglycin, cefoperazone, cefsulodin, ceftazidime, cefuroxime, cefinetazole, cefotaxime, ceftriaxone, and other known cephalosporins. Lanthionine bacteriocins that may be therapeutic partners with 2,2-Bis(4-hydroxy-3-methylphenyl)heptane include, but are not limited to, nisin, subtilin, subtilin 168, duramycin, cinnamycin, ancovenin, Pep 5, epidermin, and gallidermin. All of therapeutic partners may be used in the form of pro-drugs, esters or salts thereof. In a preferred embodiment the composition comprises 2,2-Bis(4-hydroxy-3-methylphenyl)heptane and at least one of the following boric acid, polyvinyl pyrrolidone, benzoyl peroxide, chlorhexidine, mupirocin or lanthionine bacteriocin. In another preferred embodiment the composition comprises a chelator.

When 2,2-Bis(4-hydroxy-3-methylphenyl)heptane is co-administered with a therapeutic partner, the ratio of the amount of 2,2-Bis(4-hydroxy-3-methylphenyl)heptane to the amount of the therapeutic partner may vary within a wide range. The said ratio may, for example, be from 100:1 to 1:100; more particularly, it may be, for example, from 2:1 to 1:30. The amount of the therapeutic will normally be approximately similar to the amount in which it is conventionally used per se, for example from about 50 mg, advantageously from about 62.5 mg, to about 3000 mg per unit dose, more usually about 125, 250, 500 or 1000 mg per unit dose.

It is generally advantageous to use 2,2-Bis(4-hydroxy-3-methylphenyl)heptane in admixture or conjunction with one or more therapeutic partners that can result in a synergistic effect. 2,2-Bis(4-hydroxy-3-methylphenyl)heptane and the therapeutic partner(s) can be administered separately or in the form of a single composition containing the active ingredients. 2,2-Bis(4-hydroxy-3-methylphenyl)heptane and the therapeutic partner(s) may be administered simultaneously or sequentially. Examples of simultaneous administration include where two or more compounds, compositions, or vaccines which may be the same or different, are administered in the same or different formulation or are administered separately, e.g. in a different or the same formulation but within a short time (such as minutes or hours) of each other. Examples of sequential administration include where two or more compounds, compositions or vaccines which may be the same or different are not administered together within a short time of each other, but may be administered separately at intervals of, for example, days, weeks, months or years.

Formulations of the present invention include those suitable for oral, nasal, topical, transdermal, ophthalmic, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), chewing gum, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A composition of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof, and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow, controlled or sustained release of the active ingredient therein using, for example, hydroxypropylmethyl cellulose in varying proportions to provide the desired release profile, other polymer matrices, liposomes, nanospheres and/or microspheres.

Microspheres and nanospheres for delivery of pharmaceutical substances are well known (see for example U.S. Pat. No. 5,707,644, U.S. Pat. No. 6,207,197, Kumar U. Pham Pharmaceut Sci 3:234, 2000, or Nagahara et al. Antimicrobial Agents and Chemotherapy 42:2492, 1998, all of which are hereby incorporated herewith in their entirety). Matrices used to make microspheres or nanospheres include but are not limited to chitosan, calcium phosphate, poly(L-lactic acid -co-polyglycolic acid)(PLGA), poly (L-lactic acid) (PLA), starch, gelatin, dextran, collagen, albumin, alginate, alginate-poly-L-lysine, and adipic anhydride. The selection of the composition and size for manufacturing the micro- or nano-spheres depends upon the route of administration, the desired site of preferential absorption and the desired rate of release. In a preferred embodiment 2,2-Bis(4-hydroxy-3-methylphenyl)heptane is formulated in a micro- or nano-sphere for effective absorption in nasal cavity, stomach, duodenum, or eye.

They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compositions of the invention include pharmaceutically acceptable emulsions, microemulsions, solutions, suspensions, syrups and elixirs. In addition-to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, bismuth, and tragacanth, and mixtures thereof.

Formulations of the pharmaceutical compositions of the invention for rectal or vaginal administration may be presented as a suppository, which may be prepared by mixing 2,2-Bis(4-hydroxy-3-methylphenyl)heptane with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of compositions of this invention include powders, sprays, ointments, foams, pastes, creams, lotions, gels, solutions, patches and inhalants. The active compound may be mixed with a pharmaceutically acceptable carrier, and with any preservatives, buffers, or propellants which may be required.

The ointments, pastes, creams and gels may contain, in addition to active compounds of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to compositions of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

For administration by inhalation, the compositions of th present invention for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions of this invention suitable for parenteral administration comprise 2,2-Bis(4-hydroxy-3-methylphenyl)heptane in combination with one or more pharmaceutically acceptable isotonic aqueous or nonaqueous solutions, dispersions, suspensions or emulsions, or powders which may be reconstituted into injectable solutions or dispersions just prior to use, which may contain antioxidants, buffers, bacteriostats, solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compositions of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, topical, intravenous and subcutaneous doses of the compositions of this invention for a patient, when used for the indicated effects, will range from about 0.0001 to about 100 mg per kilogram of body weight per day, more preferably from about 0.01 to about 50 mg per kg per day, and still more preferably from about 1.0 to about 100 mg per kg per day, preferably from 1 to 500 mg and most preferably from about 1 to 10 mg per kg per day. Each unit dose may be, for example, 5, 10, 25, 50, 100, 125, 150, 200 or 250 mg of 2,2-Bis(4-hydroxy-3-methylphenyl)heptane. If desired, the effective daily dose of the active compound may be administered as two, three, four, five, six or more sub-doses administered separately at appropriate intervals throughout the day, optionally, in unit dosage forms.

For use in agricultural applications, the compound or compositions of the invention is suspended in an agriculturally acceptable diluent, including but not limited to water or a fertilizer solution. To assure better adhesion of the liquid for example, in the case when the suspension is applied to the plant surface, glycerin can be added to the final diluted liquid formulation. The compounds or compositions of the invention are mixed as a dry ingredient(s) with an inert agriculturally acceptable particulate dry carrier or diluent that provides a fine powdery formulation. The agriculturally acceptable diluent is one that serves as a carrier for the low concentrations of compounds or compositions of the invention. Preferably the dry diluent is one which readily suspends in suitable diluents for administration to plants, such as water.

The formulation is applied to the plant by any of a variety of art-recognized means. For example, the formulation can be applied to the plant surface by spraying. Alternatively, the solution can be introduced injectably into a plant, for example, with a syringe, applied as a solid fertilized-like preparation for absorption by the roots at the base of a plant or a solution can be distributed at the base of a plant for root absorption. The formulation can be applied as soon as symptoms appear or prophylactically before symptoms appear. Application can be repeated.

Utility

The present invention is the result of the unexpected discovery of compositions comprising 2,2-Bis(4-hydroxy-3-methylphenyl)heptane inhibit growth, activity and/or the life of microorganisms including parasites, bacteria, algae, protozoa, fungi, viruses or prions, further including dormant and/or resistant forms such as spores and cysts. Accordingly, compositions containing 2,2-Bis(4-hydroxy-3-methylphenyl)heptane inhibit or destroy such microorganisms, and are useful as disinfectants and as antiseptics, including pharmaceutical agents for animals, especially mammals, including humans, for the treatment or prevention of diseases. In one embodiment of the invention diseases are those caused by or associated with infection by microorganisms including, but are not limited to, Streptococcus spp., Staphylococcus spp., Clostridium spp., Borrelia spp., Enterococcus spp. Propionibacterium, spp and Peptostreptococcus spp. Haemophilus spp., Pseudomonas spp., Neisseria spp., Bacillus spp. Yersinia spp. Epidermidis spp., Francisella spp. Coxiella spp., Shigella spp., Campylobacter spp., Enterococcae spp., E. coli spp., Helicobacter spp., Klebsiella spp., Moraxella spp., Chlamydia spp., retrovirus, Trichophyton spp., Microsporum spp, Mycobacteria spp. Trichomonas spp, Candida spp, Aspergillus spp. and Coccidioides spp. More preferred are infections caused by Streptococcus pyogenes, Staphylococcus aureus, methicillin resistant Staphylococcus aureus (“MRSA”), Staphylococcus epidermidis, Neisseria gonorrhoeae, Mycobacteria tuberculosis, vancomycin resistant Enterococcae (“VRE”), Helicobacter pylori, Bacillus anthracis, Chlamydia pneumoniae, Chlamydia trachomatis, HIV, Campylobacter jejuni, Propionibacterium acnes, Pseudomonas aeruginosa, Haemophilus influenzae, Candida albicans, Candida atropicalis, Francisella tularensis, Yersinia pestis, Epidermidis faecalis, Trichophyton rubrum, Trichophyton tonsurans, Trichophyton mentagrophytes, Trichophyton violaceum, Trichophyton cutaneum, Epidermophyton floccosum, Pityrosporum orbiculare, Aspergillus funigatus, Aspergillus flavus, Aspergillus niger, Coccidioides immitis, Trichomonas hominis, Trichomonas tenax, Trichomonas vaginalis, Giardia lamblia, and Toxocara canis.

The present invention is also useful in a method directed to treating infections in a host in need of such treatment, which method comprises administering a therapeutically effective amount of 2,2-Bis(4-hydroxy-3-methylphenyl)heptane. In one embodiment, the infected hosts are animals, preferably mammals, most preferably human. In another embodiment, the infected host is a plant.

Optionally, nonliving material such as but not limited to soil, surfaces, medical devices etc., may be usefully treated with the instant compositions to kill microorganisms.

The present invention is also useful in a method of treating cancer, central nervous system, cardiovascular, inflammatory, or autoimmune disease in a host in need of such treatment, which method comprises administering a therapeutically effective amount of 2,2-Bis(4-hydroxy-3-methylphenyl)heptane. In one embodiment, the hosts are animals, preferably mammals, most preferably human.

The present antimicrobial preparation can also be used in a wide variety of agriculturally beneficial species such as tobacco, vegetables including cucumber, the Cruciferae, pea and corn, beans such as soy beans, grains including cotton, rice, alfalfa, oat and other cereals, fruits, including apple, pear, peach, plum, tomato, banana, prune and citrus fruits, tubers and bulbs including potatoes and onions, nuts including walnut, grasses including sugar cane and the like.

The antibiotic preparation also is beneficial in the treatment of nursery plants and ornamental plants such as flowers, including chrysanthemum, begonia, gladiolus, geranium, carnations and gardenias.

The compositions of the instant invention also find use in the treatment of shade trees, forest trees, annual field crops and biannual field crops.

Other plant species in which the compositions of the invention can be used are Espinas, Cotoneaster, Phyrachanthas, Stranvaesis, Fraxinus, Pyrus, Malus, Capsicum, Cydonia, Crataegus and Soreus.

Besides their use as medicaments in human, veterinary or plant therapy, the compositions of the invention can also be used as animal growth promoters. For this purpose, a compound of the invention is administered orally in a suitable feed. The exact concentration employed is that which is required to provide for the active agent in a growth promotant effective amount when normal amounts of feed are consumed.

The addition of the active compound of the invention to animal feed is preferably accomplished by preparing an appropriate feed premix containing the active compound in an effective amount and incorporating the premix into the complete ration. Alternatively, an intermediate concentrate or feed supplement containing the active ingredient can be blended into the feed. The way in which such feed premixes and complete rations can be prepared and administered are described in reference books (such as such as “Applied Animal Nutrition”, W.H. Freedman and CO., S. Francisco, USA, 1969 or “Livestock Feeds and Feeding” 0 and B books, Corvallis, Oreg., USA, 1977), incorporated herein in its entirety.

Nonliving material such as but not limited to soil, porous and non-porous surfaces, etc., may be usefully treated with compositions comprising 2,2-Bis(4-hydroxy-3-methylphenyl)heptane to remove, retard or reduce the growth or, infectivity of parasites, bacteria, algae, fungi, viruses or prions including dormant and/or resistant forms such as spores and cysts, and disinfect the surface. The disinfectant compositions may be applied for example in a spray, foam or dip.

### EXAMPLE 1

2,2-Bis(4-hydroxy-3-methylphenyl)heptane

To a mixture of O-cresol (20.00 g, 0.185 mol), 2-heptanone (10.56 g, 0.0925 mol) and water (8 ml) was added dropwise 95% sulfuric acid (34 g). After being stirred for 18 hours at room temperature, the mixture was diluted with water (200 ml) and extracted with ether (100 ml×3). The ethereal phase was washed with water, dried over anhydrous sodium sulfate, filtered and evaporated. The above procedure was repeated and the combined residue was purified by flash chromatography on silica column eluted with gradient of ethyl acetate/hexane (15%-30%). The collected fraction was concentrated and the residue was mixed with hexane (50 ml) and a seed of crystal of 2,2-Bis(4-hydroxy-3-methylphenyl)heptane and cooled overnight. The solid was collected by filtration, washed with cold hexane and dried in a vacuum. The final product (17.90 g, 62%) was obtained as white powder.

Mp: 99-101° C.

C₂₁H₂₈O₂ (312.43):

MS (EI+) m/e:312; Elemental Analysis: Calcd, C: 80.73%, H: 9.19%; found, C: 80.73%, H: 9.19.

The product was also analyzed by ¹H-NMR and ¹³C-NMR. The analytic and spectra data confirm the product is the one reported as Compound 5 in Domagala et al. In Resolving the Antibiotic Paradox, ed by Rosen and Mabashery, page 269, 1998.

TABLE 1
Antibacterial activity of 2,2-Bis(4-hydroxy-3-
methylphenyl) MIC Anti-Bacterial Activity (μg/ml)

| Bacteria | MIC (ug/ml) |
|---|---|
| S. epidermidis | 2 |
| S. pneumoniae | 6 |
| S. pyogenes | 6 |
| E. faecalis | 2 |
| C. diphtheriae | 2 |
| M. smegmatis | 2 |
| M. catarrhalis | 2 |
| H. influenzae | 6 |
| H. pylori | 16 |
| S. aureus | 1 |

TABLE 2
Antibacterial activity of 2,2-Bis(4-hydroxy-3-
methylphenyl)heptane in wound healing model.

| | Efficacy in Wound |
|---|---|
| Vehicle | Healing Model |
| PEG 200 | + |
| PEG600 | + |
| PEG200/Ascorbate | + |
| Ethanol/Propylene Glycol | + |
| Beta Cyclo Dextran | − |
| Sodium lauryl sulfate/Propylene | − |
| glycol/stearylalcohol | |
| PT-Transcutol/Precirol/Petrolatum | − |

2,2-Bis(4-hydroxy-3-methylphenyl)heptane was formulated in the vehicles indicated in Table 2 and the activity in the wound healing model as described below was determined. Efficacy was evaluated by determining the reduction in bacterial load in the wound (see Table 2).

Helicobacter pylori Colonization Model

The Helicobacter gastric colonization model, using methods generally known to those skilled in the art, is employed to evaluate the antibiotic activity against H. pylori or H. felis in vivo. For example, groups of female Balb/C mice (˜6 weeks of age) are colonized, then treated with test compound (for example, one week later). Following a period of time, half of the stomach from mice are scraped and plated onto bacterial culture medium, for instance BHT agar containing antibiotics and horse serum. The plates are incubated and colonies counted to determine whether any bacteria is recovered in the gastrointestinal tract after treatment. Additionally, urease enzymatic assay, using methods generally known to those skilled in the art, is used to determine whether urease activity from Helicobacter is present. The absence of or reduction of bacteria on the culture plate or urease activity from treated mice, compared to that from non-treated mice, indicates the test substance is effective as an antibiotic against H. pylori or H. felis.

Sepsis Model

The sepsis model, using methods generally known by those skilled in the art, is used to evaluate the prophylactic antibiotic efficacy of test compounds against a number of bacteria. Basic methods include, for example, challenging mice intraperitoneally with a lethal amount of one or more bacteria, for example Staphylococcus aureus, and 7% mucin. Approximately 1 hr before or after challenge, the mice are treated by any route of administration, for example, subcutaneously, orally or intraperitoneally, with various concentrations of test compound. Vancomycin or another antibiotic is administered to a group of mice as the positive control and the placebo group of mice is administered the vehicle alone. Mortality is monitored for 96 hr. A reduction of the comparative mortalities or an increase in survival time in the various experimental groups provides evidence of efficacy of the test compound.

Wound Healing Model

The wound healing model, using methods generally known by those skilled in the art, is used to measure the efficacy of topically applied compounds in suitable vehicles against any bacteria, for example, Staphylococcus epidermis, Staphylococcus aureus, Streptococcus pyrogenes, Haemophilus influenzae, or mixtures thereof. By removing several epidermal layers by surgical procedure, a superficial wound of approximately 1 cm² was created. About 10 ⁵ cfu/20 ul of culture medium of any bacteria, for example, S aureus, was applied to the wound. The wound was occluded with a sterile plastic film and secured with an adhesive tap After 24 hours, topical therapy with the test compound, placebo ointment, a suitable positive control ointment (i.e. neomycin-polymyxin-B-bacitracin topical ointment or triclosan ointment) (twice daily) was initiated. At the appropriate time, for instance 72 hours post-infection, the wound was sampled for microbial burden. A reduction in bacterial load in the wound is evidence that the compound is efficacious.

Alternatively, the backs of mice or rabbits are shaved. Gently scraping the skin, a superficial wound is created. About 10⁴ cfu/20 ul of any bacteria, for example, S aureus, is applied to the wound. The latter is occluded with a sterile plastic film and secured with an adhesive tape. Topical therapy is employed using the above-mentioned treatment regimen. The wound is swabbed to determine the microbial load. The swab was immersed in a defined volume of diluent and dilutions were made and plated on appropriate nutritive agar plates. A reduction is bacterial load in the wound is evidence that the compound is efficacious.

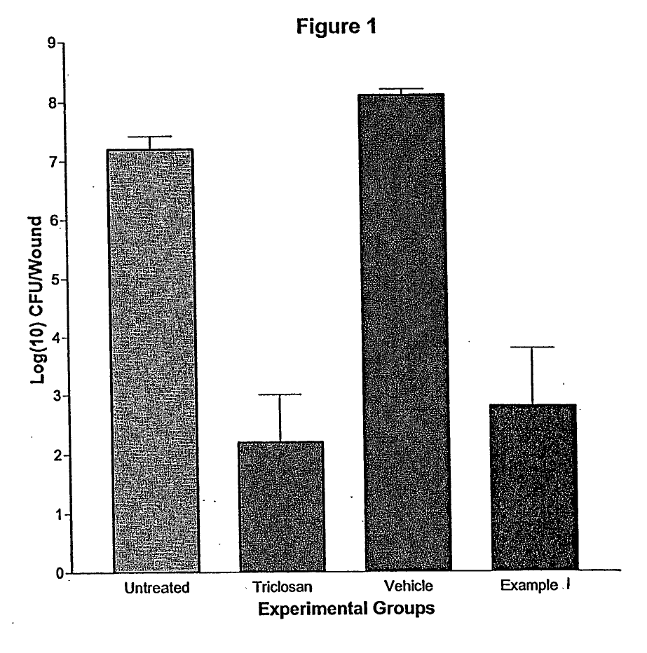

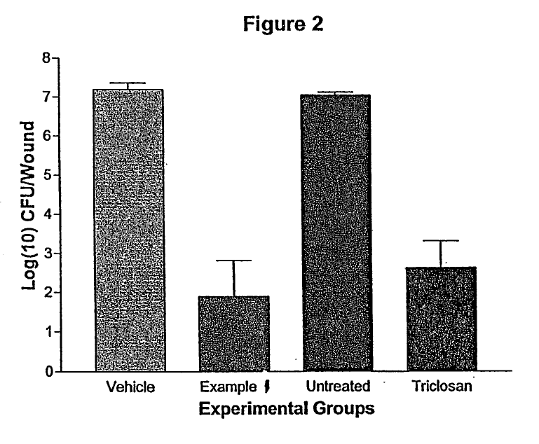

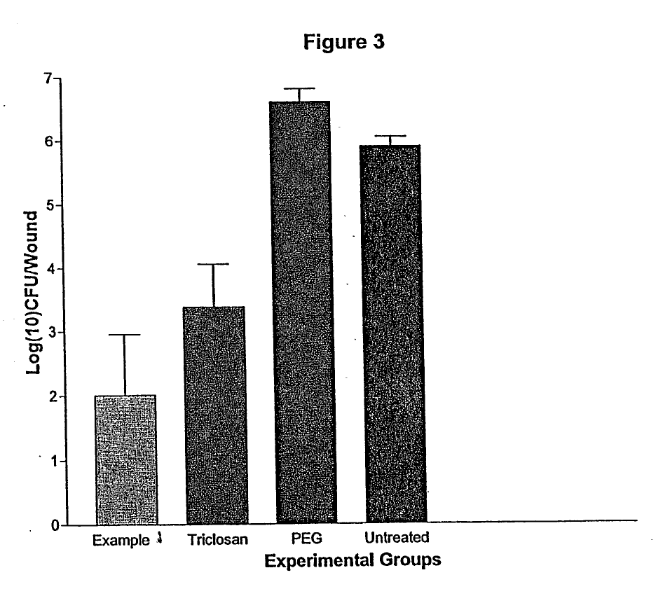

As shown in FIGS. 1-3 a composition comprising the compound of Example 9 was efficacious in reducing the population levels of pathogenic bacteria such as methicillin-susceptible and methicillin resistant Staphylococcus aureus and Staphylococcus epidermidis.

Shigella Wasting Model

Using methods well known to those skilled in the art, the Shigella sublethal wasting model is used to evaluate the antibiotic activity against Shigella flexneri or Shigella sonnei. For example, groups of mice are challenged intranasally with a sublethal wasting dose (˜10⁵ cfu) of either live Shigella flexneri or Shigella sonnei. Immediately before and at 1, 2, 5 and 7 days following challenge animals are weighed and the mean group weight determined. Approximately 1 hour after challenge, the mice are treated by any route of administration, for example, subcutaneously, orally or intravenously, with various concentrations of test compound. A suitable antibiotic is administered to a group of mice as the positive control and the placebo group of mice is administered vehicle alone. Antibiotic activity is measured by a reduction of weight loss.

Campylobacter jejuni Lethality Model

Using methods well known to those skilled in the art, the C. jejuni mortality model is used to evaluate the antibiotic activity against Campylobacter jejuni. For example, groups of mice are challenged with a single lethal dose of live C. jejuni (˜10⁸ cfu) mixed with iron dextran in endotoxin free PBS delivered intraperitoneally. Approximately 1 hour after challenge, the animals are treated by any route of administration, for example, subcutaneously, orally or intraperitoneally, with various concentrations of test compound. A suitable antibiotic is administered to a group of animals as the positive control and the placebo group of animals is administered vehicle alone. Antibiotic activity is measured by a reduction in mortality.

Campylobacter jejuni Fecal Shedding Model

Using methods well known to those skilled in the art, the C. jejuni fecal shedding model is used to evaluate the antibiotic activity against Campylobacter jejuni. For example, BALB/c mice are challenged nasally or orally with 10⁸ C. jejuni. Approximately 1 hour after challenge, the mice are treated by any route of administration, for example, subcutaneously, orally or intraperitoneally, with various concentrations of test compound. A suitable antibiotic is administered to a group of mice as the positive control and the placebo group of mice is administered vehicle alone. The duration of fecal shedding is determined by monitoring over a 9 day period. Antibiotic activity is measured by a reduction in numbers of bacteria shed.

Chlamydia pneumoniae Lung Model

Using methods well known to those skilled in the art, the Chlamydia pneumoniae lung model is used to evaluate the antibiotic activity against Chlamydia pneumoniae. For example, BALB/c are inoculated intranasally with approximately 5×10⁵ IFU of C. pneumoniae, strain AR39 in 100 μl of SPG buffer. Approximately 1 hour after challenge, the mice are treated by any route of administration, for example, subcutaneously, orally or intravenously, with various concentrations of test compound. A suitable antibiotic is administered to a group of mice as the positive control and the placebo group of mice is administered vehicle alone.

Lungs are taken from mice at days 5 and 9 post-challenge and immediately homogenized in SPG buffer (7.5% sucrose, 5 mM glutamate, 12.5 mM phosphate pH 7.5). The homogenate is stored frozen at −70° C. until assay. Dilutions of the homogenate are assayed for the presence of infectious Chlamydia by inoculation onto monolayers of susceptible cells (for example HL cells). The inoculum is centrifuged onto the cells and the cells are incubated for three days at 35° C. in the presence of 1 μg/ml cycloheximide. After incubation the monolayers are fixed with formalin and methanol then immunoperoxidase stained for the presence of Chlamydial inclusions using convalescent sera from rabbits infected with C. pneumoniae and metal-enhanced DAB as a peroxidase substrate. Antibiotic activity is measured by a reduction in numbers of Chlamydia.

Chlamydia trachomatis Infertility Model

Using methods well known to those skilled in the art, the Chlamydia trachomatis infertility model is used to evaluate the antibiotic activity against Chlamydia trachomatis. Female C3HeOuJ mice are administered a single intraperitoneal dose of progesterone (2.5 mg in pyrogen-free PBS, Depo-Provera, Upjohn) to stabilize the uterine epithelium. One week later, animals are infected by bilateral intraoviduct inoculation with approximately 5×10⁵ inclusion forming units (IFU) of C. trachomatis (including but not limited to serovar F, strain NI1) in 100 μl of sucrose phosphate glutamate buffer (SPG). At the appropriate time (for example, approximately 1 hour or 1 week after challenge), the mice are treated by any route of administration, for example, subcutaneously, orally or intravenously, with various concentrations of test compound. A suitable antibiotic is administered to a group of mice as the positive control and the placebo group of mice is administered vehicle alone. At week 3, females from each group are caged with 8-10 week old male C3H mice for a 2 month breeding period to assess fertility (1 male for every 2 females per cage with weekly rotation of the males within each group, animals from different experimental groups were not mixed). Palpation and periodic weighing are used to determine when animals in each pair become pregnant. The parameters used to estimate group fertility are: F, the number of mice which littered at least once during the mating period divided by the total number of mice in that study group; M, the number of newborn mice (born dead or alive) divided by the number of litters produced in that group during the mating period; and N, the number of newborn mice (born dead or alive) divided by the total number of mice in that group. Antibiotic activity is measured by an increase in fertility.

Chlamydia Vaginal Infection Model

Using methods well known to those skilled in the art, the Chlamydia trachomatis vaginal infection model is used to evaluate the antibiotic activity against Chlamydia trachomatis. For example, groups of mice are vaginally challenged with a dose of live C. trachomatis in endotoxin free PBS. Approximately 1 hour after challenge, the animals are treated by any route of administration, for example, subcutaneously, orally, intravaginally or intraperitoneally, with various concentrations of test compound. A suitable antibiotic is administered to a group of animals as the positive control and the placebo group of animals is administered vehicle alone. Vaginal clearance rates are determined for each group by sampling (swab) and cultivation of vaginal secretions. Antibiotic activity is measured by a reduction in the number of bacteria.

Neisseria gonorrhoeae Lethality Model

Using methods well known to those skilled in the art, the N. gonorrhoeae mortality model is used to evaluate the antibiotic activity against Neisseria gonorrhoeae. For example, groups of mice are challenged with a single lethal dose of live N. gonorrhoeae (˜10⁸ cfu) and 7% mucin in endotoxin free PBS delivered intraperitoneally. Approximately 1 hour after challenge, the animals are treated by any route of administration, for example, subcutaneously, orally or intraperitoneally, with various concentrations of test compound. A suitable antibiotic is administered to a group of animals as the positive control and the placebo group of animals is administered vehicle alone. Antibiotic activity is measured by a reduction in mortality.

Neisseria gonorrhoeae Vaginal Challenge Model

Using methods well known to those skilled in the art, the N. gonorrhoeae vaginal infection model is used to evaluate the antibiotic activity against Neisseria gonorrhoeae. For example, groups of mice are vaginally challenged with a dose of live N. gonorrhoeae in endotoxin free PBS. Approximately 1 hour after challenge, the animals are treated by any route of administration, for example, subcutaneously, orally or intraperitoneally, with various concentrations of test compound. A suitable antibiotic is administered to a group of animals as the positive control and the placebo group of animals is administered vehicle alone. Vaginal clearance rates are determined for each group by sampling (swab) and cultivation of vaginal secretions. Antibiotic activity is measured by a reduction in the number of N. gonorrhoeae.

Giardia Model

Compounds are tested for in vitro growth inhibitory activity against Giardia lamblia using methods described in Katiyar, S. K. and Edind,T. D., Antimicroorganisms and Chemotherapy, vol. 35, pp. 2198-2202 (1991) which is incorporated herein in its entirety. EC50 values for test compounds, expressed in parts per million (ppm), were calculated from dose response curves. As used herein, the terminology “EC50” means the concentration of test compound required to inhibit growth by 50% are compared to a control lacking the test compound. A suitable antibiotic, for instance, Metronidazole is included as a standard in the tests for comparative purposes.

Ophthalmic Infection Model

Anesthetized animals are inoculated with the appropriate number of Staphylococcus aureus or Pseudomonas aeruginosa colony forming units. After approximately 24 hours, animals are treated by any means, for instance topically, orally, intravenously, or intraperitoneally, with formulations of various concentrations of test antibiotics at various frequencies for 1 to 2 days. Corneas are excised from euthanized animals and are homogenized. The homogenate is plated onto bacterial culture medium, for instance trypticase soy agar plates. The plates are incubated and colonies counted to determine whether any bacteria is recovered. The absence of or reduction of bacteria on the culture plate treated animals, compared to that from non-treated animals, indicates the test substance is effective as an antibiotic against the test organism.

Candida albicans Skin Infection Model

Three days before infection, suitable numbers of mice are treated i.p. with cyclophosphamide (approximately 150 mg/kg). On Day 0, the backs of mice are shaved. Gently scraping the skin, a superficial wound is created. About 10⁶ cfu of Candida albicans in approximately 0.1 ml is applied to the wound. The wound is occluded with a sterile plastic film and secured with an adhesive tape. Beginning approximately 30 hours after the yeast challenge, the test material is applied topically once a day, twice a day or three times a day for five consecutive days. An appropriate anti-fungal (i.e. clotrimazole) is administered a various frequencies, for instance twice a day, to one group of mice as a positive control. Two days after the last treatment, the mice are sacrificed and the treated area excised and homogenized in sterile saline. The homogenate is diluted and plated on appropriate agar plates (i.e. Sabauroud's agar plates+selective antibiotics and incubated at the appropriate temperature A reduction is bacterial load in the wound is evidence that the compound is efficacious.

Trichomonas Model

Representative tests of the activity of test compound against Trichomonas vaginalis is carried out by the two-fold tube dilution method. Trichomonas vaginalis is grown on Trichomonas Culture Medium Base (Merck) plus 10% horse serum approximately 10⁵ organisms/ml and incubated under the appropriate conditions. The ability of the test compound to act as an antimicrobial is determined by the ability to inhibit growth of Trichomonas vaginalis.

Diabetic Wound Model

Rats or mice are rendered diabetic by an injection of streptozotocin. Approximately 6 weeks after injection of streptozotocin, excisional wounds are made on the abdomen or thighs. Animals are anesthetized and hair from the wound area removed. Wound areas are marked using a template and the area of skin is removed to the depth of the muscle fascia using dissecting scissors and forceps. The wounds are treated with test compound or appropriate positive control antibiotic at various frequencies. Wound area is measured at various days. A reduction in the wound area in treated versus non-treated controls is evidence of efficacy.

While the preferred embodiments of the invention have been illustrated and described, it will be appreciated that various changes can be made therein without departing from the spirit and scope of the invention.

What is claimed is:

1. A method of treating or preventing a disease or condition caused by or associated with a microbial infection, which method comprises the administration to a patient in need thereof a pharmaceutical composition comprising an anti-microbial amount of 2,2-Bis(4-hydroxy-3-methylphenyl)heptane and a carrier.

2. The method of claim 1 wherein the composition further comprises one or more agents selected from the group consisting of antibiotics, steroids, vaccines, anti-oxidants, zinc chloride, ascorbic acid, ascorbate, dextran sulfate, non-steroidal anti-inflammatories, antacids, antibodies, chelators, benzoyl peroxide, boric acid, polyvinyl pyrrolidone, interferons, proteases, glucosidases, pectinases, amylases, lipases and cytokines

3. The method of claim 1 wherein the composition is administered to the skin, mouth, eye, respiratory tract, urinary tract, or reproductive tract of an animal.

4. The method of claim 1 wherein the composition is administered to a mucosal surface of an animal.

5. The method of claim 2 wherein the composition is administered to the skin, mouth, eye, respiratory tract, urinary tract, or reproductive tract of an animal.

6. The method of claim 2 wherein the composition is administered to a mucosal surface of an animal.

7. The method of claim 1 when the composition is administered to the skin, mouth, eye, respiratory tract, urinary tract, or reproductive tract of a human.

8. The method of claim 1 wherein the composition is administered to a mucosal surface of a human.

9. The method of claim 2 wherein the composition is administered to the skin, mouth, eye, respiratory tract, urinary tract, or reproductive tract of a human.

10. The method of claim 2 wherein the composition is administered to a mucosal surface of a human.

11. The method of claim 1 wherein the composition is administered topically

12. The method of claim 2 wherein the composition is administered topically.

13. The method of claims 1, 2, 11 or 12 wherein the composition comprises polyethylene glycol.

14. The method of claims 1, 2, 11 or 12 wherein the composition comprises polyethylene glycol 200 or polyethylene glycol 600.

15. The method of claims 1, 2, 11, or 12 wherein the composition comprises ascorbate or ascorbic acid.

16. The method of claim 1 wherein the disease or condition is caused by or associated with infection with a microbe selected from the group consisting of Streptococcus spp., Staphylococcus spp., Clostridium spp., Borrelia spp., Enterococcus spp. Propionibacterium, spp and Peptostreptococcus spp. Haemophilus spp., Pseudomonas spp., Neisseria spp., Bacillus spp. Yersinia spp. Epidermidis spp., Francisella spp. Coxiella spp., Shigella spp., Campylobacter spp., Enterococcae spp., E. coli spp., Helicobacter spp., Klebsiella spp., Moraxella spp., Chlamydia spp., retrovirus, Trichophyton spp., Microsporum spp, Mycobacteria spp. Trichomonas spp, Candida spp, Aspergillus spp. and Coccidioides spp.

17. The method of claim 1 wherein the disease or condition is caused by or associated with infection with a microbe selected from the group consisting of Streptococcus pyogenes, Staphylococcus aureus, methicillin resistant Staphylococcus aureus (“MRSA”), Staphylococcus epidermidis, Neisseria gonorrhoeae, Mycobacteria tuberculosis, vancomycin resistant Enterococcae (“VRE”), Helicobacter pylori, Bacillus anthracis, Chlamydia pneumoniae, Chlamydia trachomatis, HIV, Campylobacter jejuni, Propionibacterium acnes, Pseudomonas aeruginosa, Haemophilus influenzae, Candida albicans, Candida atropicalis, Francisella tularensis, Yersinia pestis, Epidermidis faecalis, Trichophyton rubrum, Trichophyton tonsurans, Trichophyton mentagrophytes, Trichophyton violaceum, Trichophyton cutaneum, Epidermophyton floccosum, Pityrosporum orbiculare, Aspergillus funigatus, Aspergillus flavus, Aspergillus niger, Coccidioides immitis, Trichomonas hominis, Trichomonas tenax, Trichomonas vaginalis, Giardia lamblia, and Toxocara canis.

18. The method of claim 2 wherein the disease or condition is caused by or associated with infection with a microbe selected from the group consisting of Streptococcus spp., Staphylococcus spp., Clostridium spp., Borrelia spp., Enterococcus spp. Propionibacterium, spp and Peptostreptococcus spp. Haemophilus spp., Pseudomonas spp., Neisseria spp., Bacillus spp. Yersinia spp. Epidermidis spp., Francisella spp. Coxiella spp., Shigella spp., Campylobacter spp., Enterococcae spp., E. coli spp., Helicobacter spp., Klebsiella spp., Moraxella spp., Chlamydia spp., retrovirus, Trichophyton spp., Microsporum spp, Mycobacteria spp. Trichomonas spp, Candida spp, Aspergillus spp. and Coccidioides spp.

19. The method of claim 2 wherein the disease or condition is caused by or associated with infection with a microbe selected from the group consisting of Streptococcus pyogenes, Staphylococcus aureus, methicillin resistant Staphylococcus aureus (“MRSA”), Staphylococcus epidermidis, Neisseria gonorrhoeae, Mycobacteria tuberculosis, vancomycin resistant Enterococcae (“VRE”), Helicobacter pylori, Bacillus anthracis, Chlamydia pneumoniae, Chlamydia trachomatis, HIV, Campylobacter jejuni, Propionibacterium acnes, Pseudomonas aeruginosa, Haemophilus influenzae, Candida albicans, Candida atropicalis, Francisella tularensis, Yersinia pestis, Epidermidis faecalis, Trichophyton rubrum, Trichophyton tonsurans, Trichophyton mentagrophytes, Trichophyton violaceum, Trichophyton cutaneum, Epidermophyton floccosum, Pityrosporum orbiculare, Aspergillus funigatus, Aspergillus flavus, Aspergillus niger, Coccidioides immitis, Trichomonas hominis, Trichomonas tenax, Trichomonas vaginalis, Giardia lamblia, and Toxocara canis.

20. The method of claim 1 or 2 wherein 2,2-Bis(4-hydroxy-3-methylphenyi)heptane is administered in a unit dose of from about 0.1 mg to about 3000 mg.

21. The method of claim 20, wherein the unit dose is about 5 to 500 mg.

22. The method of claim 20, wherein the unit dose is selected from the group consisting of 5, 10, 25, 50, 100, 125, 150, 200, 250, and 500 mg.

23. The method of claim 1 or 2 wherein the composition is administered two or more times.

24. The method-of claim 1, 2 or 23 wherein the dose of 2,2-Bis(4-hydroxy-3-methylphenyl)heptane administered is about 0.0001 to about 100 mg per kilogram of body weight per day

25. The method of claim 1, 2, or 23 wherein the dose of 2,2-Bis(4-hydroxy-3-methylphenyl) administered is from about 0.01 to about 50 mg per kg per day.

26. The method of claim 1,2 or 23 wherein the dose of 2,2-Bis(4-hydroxy-3-methylphenyl)heptane administered is from about 0.1 to about 10 mg per kg per day

27. The method of claim 1, 2, or 23 wherein the dose of 2,2-Bis(4-hydroxy-3-methylphenyl)heptane administered is from 5 to 500 mg.

28. The method of claim 1, 2 or 23 wherein the dose of 2,2-Bis(4-hydroxy-3-methylphenyl)heptane administered is selected from the group consisting of 5, 10, 25, 50, 100, 125, 150, 200, 250 and 500 mg per kg per day.

29. A method of cleaning and disinfecting an inert or living surface at least partly covered by a biofilm layer by contacting the biofilm with a composition comprising 2,2-Bis(4-hydroxy-3-methylphenyl)heptane in an amount effective for either fully or partly removing or releasing the biofilm layer and a carrier.

30. A method of inhibiting the formation of a biofilm on an inert or living surface comprising contacting a surface with an inhibiting effective amount of a composition comprising 2,2-Bis(4-hydroxy-3-methylphenyl)heptane and a carrier.

31. The method of claim 30 wherein the surface is a mucous membrane.

32. A method of cleaning and disinfecting an inert or living surface at least partly covered by a biofilm layer by contacting the biofilm with 2,2-Bis(4-hydroxy-3-methylphenyl)heptane in an amount effective for either fully or partly removing or releasing the biofilm layer.

33. A method of inhibiting the formation of a biofilm on an inert or living surface comprising contacting a surface with an inhibiting effective amount of 2,2-Bis(4-hydroxy-3-methylphenyl)heptane.
